(19) United States
(12) Patent Application Publication
(10) Pub. No.: US 2006/0142780 A1
(43) Pub. Date: Jun. 29, 2006

(54) Preloaded IOL injector and method

(76) Inventors: Joel Pynson, Toulouse (FR); Benoit Bessiere, Dremil Lafage (FR); Brian D. Rathert, St. Petersburg, FL (US)

Correspondence Address: Bausch & Lomb Incorporated, One Bausch & Lomb Place, Rochester, NY, 14604-2701, US

(21) Appl. No.: 11/025,406

(22) Filed: Dec. 29, 2004

(51) Int. Cl. A61F 9/00

(52) U.S. Cl. 606/107

(57) ABSTRACT

Apparatus and method for preloading an intraocular lens in a component of an injector device. The intraocular lens is positioned in a shuttle which is positioned inside a distal section of the device. The shuttle, IOL and distal section are positioned and sealed in a vial of storage solution. At the time of use, the surgeon opens the vial and attaches a proximal section having a plunger to the distal section located in the vial. The proximal section is then lifted away from the vial together with the distal section, and the shuttle and IOL located in the distal section. The plunger is advanced to express the IOL from the distal tip of the distal section.

### BACKGROUND OF THE INVENTION

The present invention relates to ophthalmic surgical devices and methods. More particularly, the present invention relates to a device and method for inserting an intraocular lens (IOL) into an eye wherein the IOL may be conveniently preloaded in and packaged together with the injector device.

IOLs are artificial lenses used to replace the natural crystalline lens of the eye when the natural lens has cataracts or is otherwise diseased. IOLs are also sometimes implanted into an eye to correct refractive errors of the eye in which case the natural lens may remain in the eye together with the implanted IOL. The IOL may be placed in either the posterior chamber or anterior chamber of the eye. IOLs come in a variety of configurations and materials. Some common IOL styles include the so-called open-looped haptics which include the three-piece type having an optic and two haptics attached to and extending from the optic; the one-piece type wherein the optic and haptics are integrally formed (e.g., by machining the optic and haptics together from a single block of material); and also the closed looped haptic IOLs. Yet a further style of IOL is called the plate haptic type wherein the haptics are configured as a flat plate extending from opposite sides of the optic. The IOL may be made from a variety of materials or combination of materials such as PMMA, silicone, hydrogels and silicone hydrogels, etc.

Various instruments and methods for implanting the IOL in the eye are known. In one method, the surgeon simply uses surgical forceps having opposing blades which are used to grasp the IOL and insert it through the incision into the eye. While this method is still practiced today, more and more surgeons are using more sophisticated IOL inserter devices which offer advantages such as affording the surgeon more control when inserting the IOL into the eye. IOL inserter devices have recently been developed with reduced diameter insertion tips which allow for a much smaller incision to be made in the cornea than is possible using forceps alone. Smaller incision sizes (e.g., less than about 3 mm) are preferred over larger incisions (e.g., about 3.2 to 5+mm) since smaller incisions have been attributed to reduced post-surgical healing time and complications such as induced astigmatism.

Since IOLs are very small and delicate articles of manufacture, great care must be taken in their handling. In order for the IOL to fit through the smaller incisions, they need to be folded and/or compressed prior to entering the eye wherein they will assume their original unfolded/uncompressed shape. The IOL inserter device must therefore be designed in such a way as to permit the easy passage of the TOL through the device and into the eye, yet at the same time not damage the delicate IOL in any way. Should the IOL be damaged during delivery into the eye, the surgeon will most likely need to extract the damaged IOL from the eye and replace it with a new IOL, a highly undesirable surgical outcome.

Thus, as explained above, the IOL inserter device must be designed to permit easy passage of the IOL therethrough. It is equally important that the IOL be expelled from the tip of the IOL inserter device and into the eye in a predictable orientation and manner. Should the IOL be expelled from the tip too quickly or in the wrong orientation, the surgeon must further manipulate the IOL in the eye which could result in trauma to the surrounding tissues of the eye. It is therefore highly desirable to have an inserter device which allows for precise loading of the IOL into the inserter device and which will pass and expel the IOL from the inserter device tip and into the eye in a controlled, predictable and repeatable manner.

To ensure controlled expression of the IOL through the tip of the IOL inserter device, the IOL must first be loaded into the IOL inserter device.

The loading of the IOL into the inserter device is therefore a precise and very important step in the process. Incorrect loading of an IOL into the inserter device is oftentimes cited as the reason for a failed IOL delivery sequence. Many 101 injector devices on the market today require the IOL to be loaded into the injector at the time of surgery by the attending nurse and/or surgeon. Due to the delicate nature of the IOL, there is a risk that the nurse and/or surgeon will inadvertently damage the IOL and/or incorrectly load the TOL into the injector device resulting in a failed implantation. Direct handling and/or loading of the IOL into the injector by the nurse and/or surgeon is therefore undesirable.

There remains a need for an IOL inserter and method which removes the need for direct handling of the IOL by the nurse and/or surgeon and which generally simplifies operation of the IOL injector device and IOL delivery process.

### SUMMARY OF THE INVENTION

In a broad aspect of the invention, an injector device is provided having proximal and distal sections which are packaged separately and then assembled together at the tine of surgery. The injector device provides an IOL preloaded in the distal section of the device which is stored and packaged either in a dry package or submersed in a sterile storage solution. At the time of surgery, the nurse or surgeon simply opens the packages containing the distal and proximal sections and attaches the proximal and distal sections together. The injector device is then ready to deliver the IOL from the injector device and into an eye. No other injector components are required to ready the device for delivery of the IOL therethrough.

At the injector device manufacturing site, an IOL is placed in the distal section of the device which is placed in a dry package or a container (e.g. a vial) of hydrating solution and sealed. Particularly, the distal section includes a shuttle component having an IOL loading area in which the IOL is placed, preferably in an unstressed condition, i.e., in a condition where at least the IOL optic is not compressed or folded. The shuttle and IOL are inserted into a nozzle section which includes a distal tip through which the IOL is ultimately expelled from the injector device. The shuttle and nozzle each include a longitudinal passageway which preferably lie along a common longitudinal axis when the shuttle and nozzle sections are assembled together. The assembled shuttle (with IOL placed therein) and nozzle section together comprise the distal section of the injector device which is placed either in a dry package or in a vial of hydrating solution (e.g., buffered saline). The package or vial is then sealed and sterilized. The hydrating solution maintains the IOL in a hydrated state during shipping and storage, a necessary requirement for IOLs made of certain materials such as hydrogels.

The proximal section of the injector device is provided in a separately sealed and sterilized package although the proximal section and package or vial containing the distal section may be provided in a single “kit” type package if desired for sake of convenience to the user. The proximal section of the injector device includes a tubular body having a longitudinal passageway extending between opposite, open ends thereof. A plunger component is inserted into the proximal open end of the tubular body and telescopes within the longitudinal passageway thereof. The plunger includes a finger press at the proximal end thereof for manually pressing and advancing the plunger through the passageway, and a plunger tip at the opposite, distal end thereof for engaging and pushing the IOL through and out the distal tip of the nozzle section of the injector device.

At the time of surgery, the nurse or surgeon removes the outer packaging from the proximal section of the device and opens the package or vial containing the distal section of the device. The distal, open end of the proximal section is inserted into the open end of the package or vial with the proximal section being snapped onto the distal section of the injector device. With the proximal and distal sections thus attached together, the proximal section is lifted away from the package or vial and thereby also removing the distal section from the vial. The package or vial and hydrating solution may then be discarded or recycled. With the proximal and distal sections of the device attached together, the device is ready to be used to implant the IOL into a patient's eye. No further attachment or removal of injector component parts is necessary as is required in more complicated prior art devices.

The injector device includes means for compressing or otherwise urging the IOL into a smaller cross-section for delivery through the injector. In a preferred embodiment of the invention, the shuttle and nozzle passageways are configured with a narrowing taper towards the distal tip. The plunger is advanced at the proximal end of the injector device causing the distal tip of the plunger to engage the IOL optic. As the plunger is advanced further, the IOL is pushed through the narrowing passageway, thereby compressing the IOL into a smaller cross-section and finally exiting at the distal end of the injector body and expressed into the eye in the intended manner.

The relative positioning of the IOL shuttle, the IOL and the injector device is such that upon attaching the proximal and distal sections of the injector device together, the IOL becomes preferentially positioned inside the injector device. The IOL thus becomes positioned in a particular orientation inside the injector device relative to the plunger tip. This IOL loaded position results in the leading haptic being correctly aligned in the shuttle, and the trailing haptic and optic aligning with the plunger tip so that upon advancement of the plunger, the plunger tip will engage the IOL optic in the intended manner without obstruction or jamming of the trailing haptic.

### BRIEF DESCRIPTION OF THE DRAWINGS

FIG. 1 is an exploded, side elevational view of a first embodiment of the injector device showing main components of injector device;

FIG. 2 is a perspective view of the fully assembled injector device of FIG. 1 showing the an IOU expressed from the distal tip thereof;

FIG. 3a is an enlarged perspective view of the proximal section of the injector device of FIGS. 1 and 2;

FIG. 3b is a side elevational view thereof;

FIG. 3c is an end view thereof;

FIG. 4a is a side elevational view of the plunger component of the injector device;

FIG. 4b is a perspective view thereof;

FIG. 5a is a perspective, top view of a first embodiment of the shuttle component of the injector device with IOL loading area in the open is position and an IOL positioned therein;

FIG. 5b is a perspective view of the shuttle component of FIG. 5a with the IOL loading area shown in the closed position and the shuttle being rotated 180o from the position shown in FIG. 5a;

FIG. 5c is a top plan view of the shuttle component of FIGS. 5a,b with the IOL loading area in the open position;

FIG. 5d is an end view of FIG. 5c taken from the left side thereof;

FIG. 6a is a perspective view of the distal section of the injector device of the previous Figures;

FIG. 6b is a top plan view thereof;

FIG. 6c is a side elevational thereof;

FIG. 6d is an end view thereof;

FIG. 7 is a perspective view of the distal section and shuttle component positioned in a first embodiment of the vial component of the invention;

FIG. 8a is a side elevational view of the proximal section and plunger components of the injector device in the process of coupling to the distal section located in the vial;

FIG. 8b is a perspective view thereof;

FIG. 9a is a perspective view of a second embodiment of the shuttle component with the IOL loading area in the open position and another style of IOL positioned therein;

FIG. 9b is the view of FIG. 9a with the shuttle IOL loading area in the closed position;

FIG. 9c is the view of FIG. 9b with a second embodiment of the distal section of the device shown coupled to the shuttle; and

FIG. 10 is a perspective view of the first embodiment of the distal section shown coupled to the first embodiment of the shuttle component.

### DETAILED DESCRIPTION

In a first, broad aspect, the invention comprises a preloaded injector device for injecting an IOL into an eye. The term “preloaded” as used herein means that a packaged component of the injector device includes an IOL positioned therein. Direct handling and loading of an IOL into the injector device is therefore not necessary.

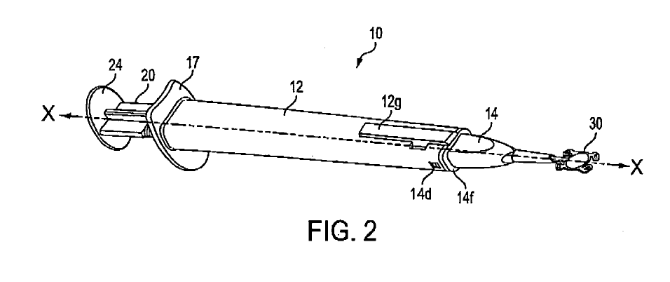

The injector device 10 includes a proximal section 12 and a distal section 14 which are packaged separately and then attached together at the time of surgery to ready the device for delivery of an IOL 30, 30′ therethrough and into a patient's eye (see FIGS. 2, 5a and 9a). The IOL 30, 30′ is preloaded into the distal section 14 of the device which is packaged in either a dry state or in a hydrated state in a vial 11 containing a storage solution to maintain the IOL in a hydrated state during shipping and storage. Whether the IOL is packaged and stored in the dry or wet state depends on the type of material from which the IOL is made. Examples of IOL materials which may be packaged in the dry state include silicone while IOL materials which require wet storage include acrylic.

The proximal section 12 includes a longitudinal passageway 12a extending between the open proximal and distal ends 12b, 12c thereof, respectively. The passageway 12a may assume any desired cross-sectional shape such as a rounded rectangular shape as shown.

The distal section 14 includes a longitudinal passageway 14a extending between the open proximal end 14b and open distal tip 15c thereof. The passageway 14a tapers inwardly toward distal tip 14c so that the IOL is gradually compressed to a very small cross-section as it exits the device at tip 15c.

A first embodiment of the shuttle component 16 is provided into which an IOL 30 is loaded and held in an uncompressed condition. This will be described in more detail below. Shuttle 16, with an IOL 30 loaded therein, is positioned in distal section passageway 14a. Shuttle 16 also includes a longitudinal passageway 16a extending between the open proximal end 16b and open distal end 16c thereof. When shuttle 16 is positioned in distal section 14, it is preferred, though not necessary, that the longitudinal passageways 16a, 14a of each are aligned along the same axis X-X. When the proximal section 12 is attached to the distal section 14, the longitudinal passageway 12a is aligned along the common axis X-X of the distal and shuttle passageways 14a, 16a (FIG. 2).

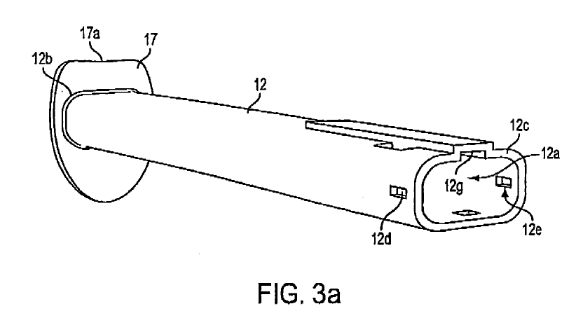

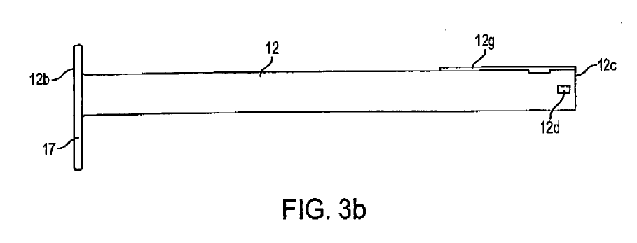

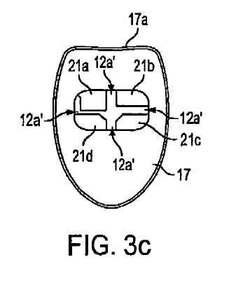

Referring again to proximal section 12, a finger flange 17 may be formed at the proximal end 12b thereof for ease in operating the injector device in the manner of a syringe. Finger flange is preferably configured with a straight edge 17a as shown (FIG. 3a) for resting device 10 on a flat surface.

A plunger 20 having proximal and distal lengths 20a, 20b, respectively, a distal plunger tip 22, and a thumb press 24 telescopes within the proximal section 12. When the proximal and distal sections 12, 14 are attached together, the plunger 20 extends sequentially through proximal section passageway 12a and the shuttle passageway 16a so as to engage and push the IOL 30 through passageway 16a and out distal to tip 15c. The IOL delivery sequence will be explained in more detail below.

It is understood that the overall configuration of the injector body 12 may vary from that shown and described herein. It is furthermore understood that the components of the injector device may be made of any suitable material (e.g., polypropylene) and may be wholly or partly opaque, transparent or translucent to better visualize the IOL within the injector device and the IOL delivery sequence. In a preferred embodiment of the injector device, the components thereof are steam sterilized, requiring that the components are made from a material which can withstand the heat generated during steam sterilization.

Examples of such materials include, but are not limited to, polypropylene, polycarbonate, polysulfone, ALTEM (by Dupont), and PFA.

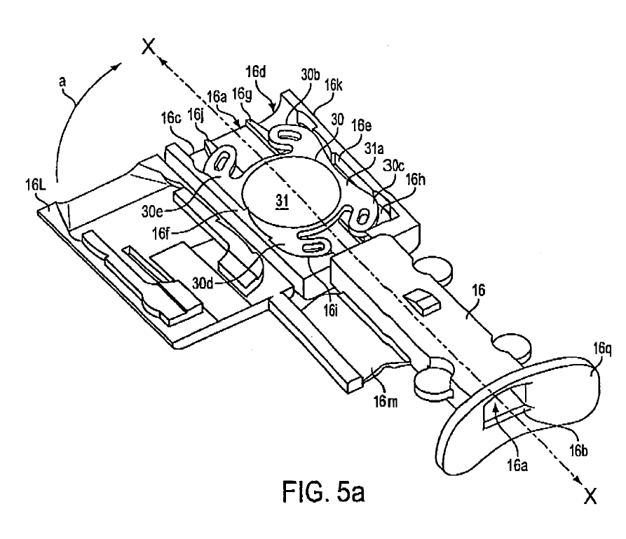

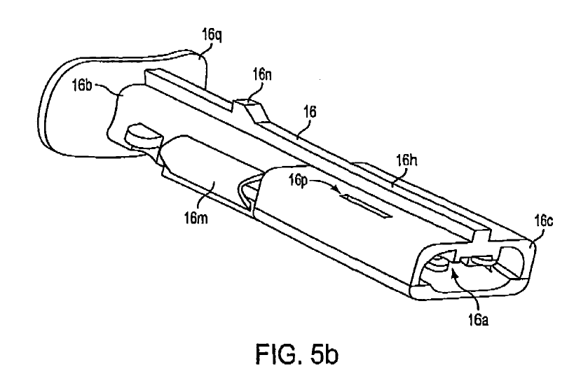

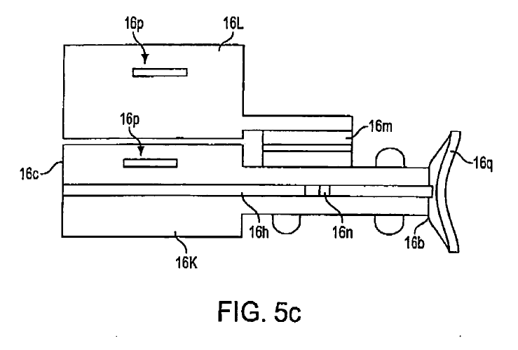

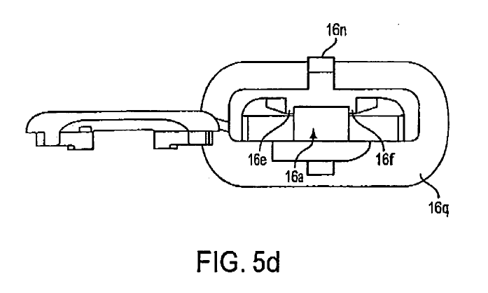

A first embodiment of shuttle 16 is used for holding an IOL 30 in the preloaded position. The shuttle 16, with IOL 30 held thereby, is positioned in the distal section 14 through opening 14a thereof. As seen best in FIGS. 5a-d, shuttle 16 includes an IOL loading area 16d wherein the IOL 30 is positioned in an unstressed state. Loading area 16d is in open communication with longitudinal passageway 16a and is configured to position the IOL 30 along axis X-X in an unstressed state and may include one or more optic support elements 16e,f each having a radius or other feature for aligning the IOL optic 31 along passageway 16a (and hence also axis X-X) about the periphery 31a thereof.

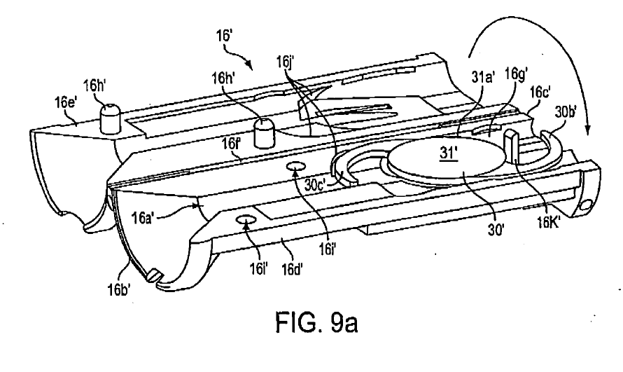

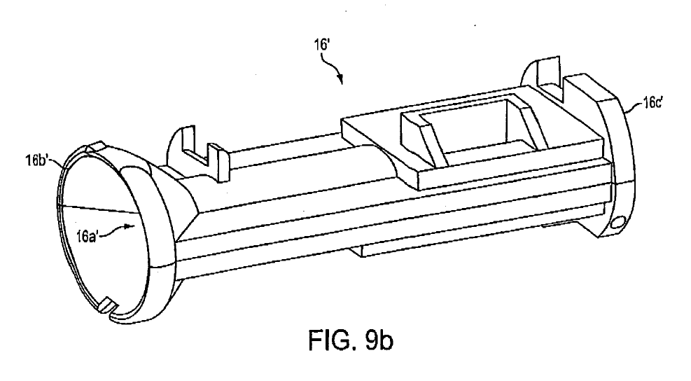

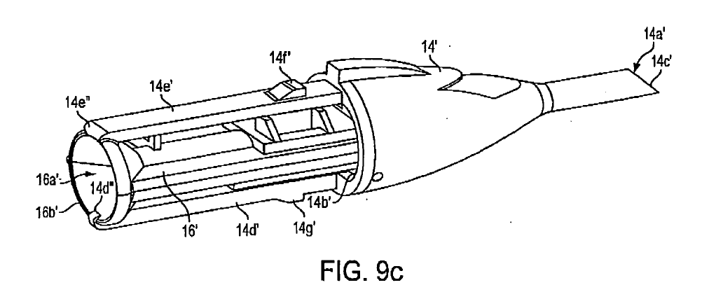

Alternatively or in addition to the optic support elements, one or more haptic support elements 16g-j are provided on shuttle 16, each of which include a radius or other feature for aligning one or more haptics 30b-e which attach to and extend from the optic 31. In this regard, it is understood that the two IOL configurations 30, 30′ shown and described herein is for discussion purposes only, and that the present invention is not to be limited thereby. The invention may be easily adapted to IOLs of any configuration and type (e.g., IOLs with plate, open or closed loop haptics, anterior chamber IOLs, posterior chamber IOLs, accommodating IOLs (including single and double lens types), etc.). The overall configuration of the TOL shuttle 16 and IOL loading area 16a may thus likewise vary so as to be cooperatively configured with and align the particular IOL style being used with the device. For ease of description, the first invention embodiment will be described with reference to IOL 30. In all embodiments, the shuttle 16 holds at least the IOL optic 31 in the unstressed state. It is furthermore preferable that shuttle 16 hold the IOL haptics at the correct vault angle (i.e., the angle from which they normally extend from the IOL optic periphery). It is even furthermore preferable that, in the case of an IOL having open looped haptics, such as IOL 30′ seen in FIG. 9a, the haptic support elements maintain the looped haptics 30b′, 30c′ at the correct angle of curvature. In FIGS. 5a and 9a, it is seen that the haptic support elements constrain the haptics along the outer curved edges thereof. This ensures that the haptic curvature, which is designed and set at manufacture of the haptics, does not increase or bend out of specification during storage of the IOL and shuttle. The embodiment of FIGS. 9a-c will be described more fully below.

At manufacture, the IOL 30 is placed in the shuttle 16. Positioning the TOL 30 in the shuttle 16 may be done by a worker using a pair of tweezers, for example, although other methods may be used as desired, including automated or semi-automated means in an assembly line. To facilitate loading of the IOL in the shuttle, the IOL loading area 16a may be formed with two wall sections 16k and 16L which are pivotally connected (e.g., via a living hinge 16m) to enable opening and closing of the IOL loading area 161. Wall sections 16k and 16L are spread open in a coplanar relationship in the open position of the shuttle loading area 16d. In this open position, IOL loading area 16d is easily accessible and an IOL 30 may be simply placed upon one of the two sections, preferably upon section 16k. This may be done by aligning the IOL optic 31 with the IOL supporting elements 16g,j and aligning the haptics 30b-e with the haptic support elements 16d, 16e, respectively.

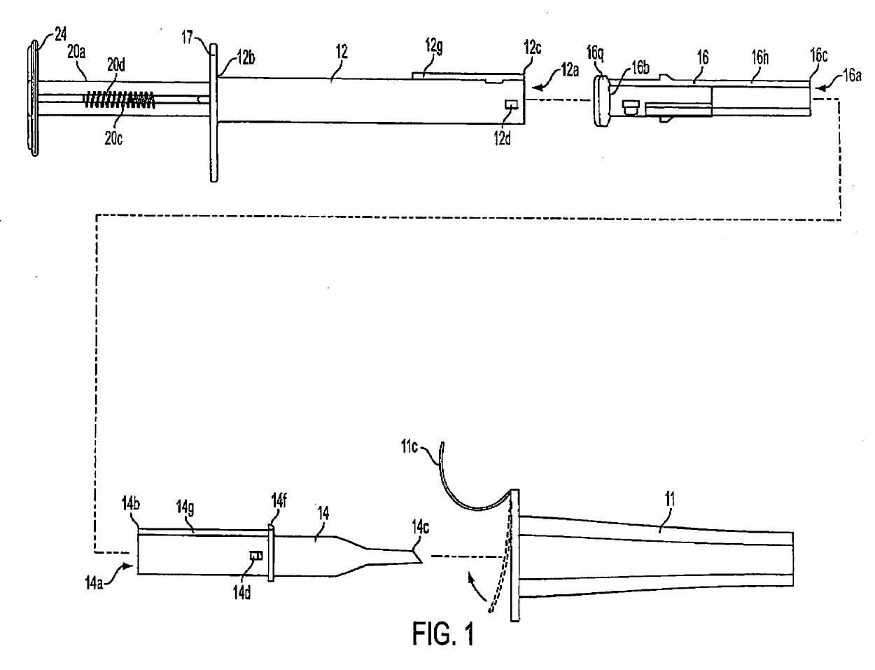

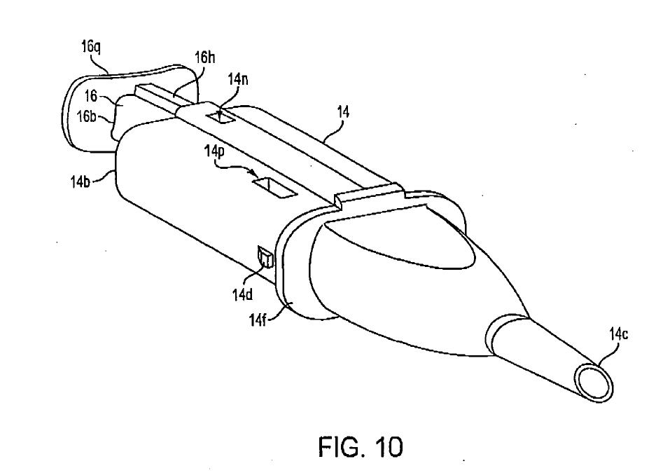

Once the IOL 30 is properly positioned in the shuttle IOL loading area 16a, the two sections 16g, 16h are pivoted together (in the direction of arrow “a” in FIG. 5a) to the closed position which encases IOL 30 between the now facing wall sections 16k, 16L (FIG. 5b). With the IOL 30 thus positioned in the shuttle 16, the shuttle 16 is closed and is then inserted into the distal section passageway 15a as seen in FIG. 1 while FIG. 10 shows the distal section 14 and shuttle 16 attached together. When fully coupled together, the proximal end 16b of the shuttle extends outwardly of the proximal end 14b of the distal section.

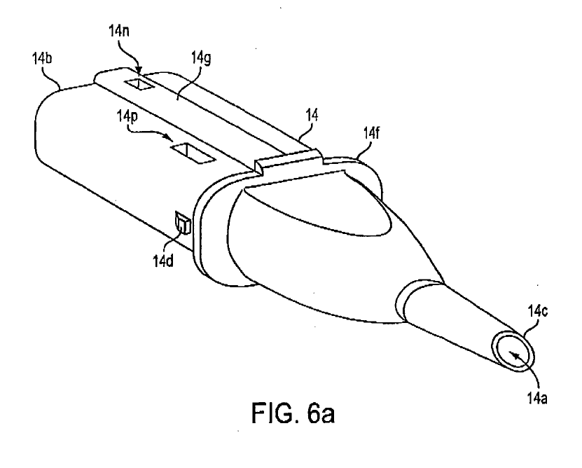

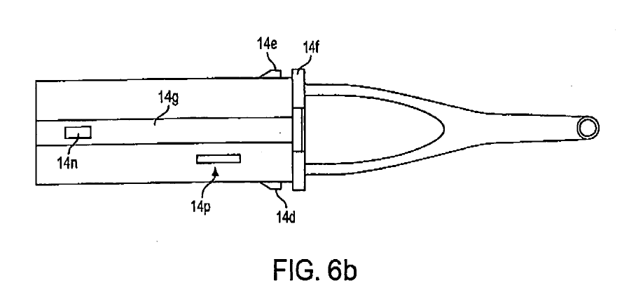

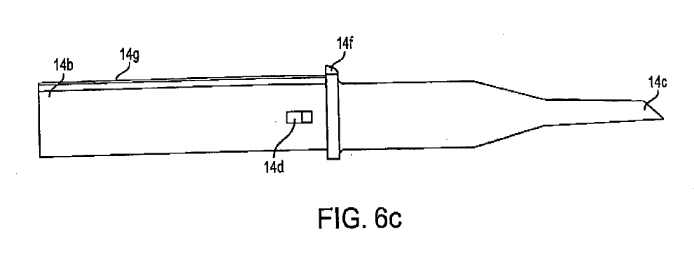

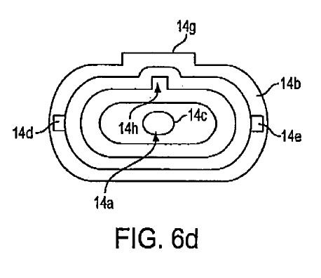

To assist in attaching the shuttle to the distal section in the correct manner, a longitudinal groove 14h (FIG. 6d) may be formed on an inner wall surface of distal section 14 which aligns with a longitudinal flange 16h formed on an outer wall surface of shuttle 16 (FIG. 5b). As such, the shuttle 16 may be slidingly received within distal section 14 with groove 144h and flange 16h providing a “key” to prevent incorrect coupling between the shuttle and distal section. Furthermore, the shuttle 16 and distal section 14 may be fixed in the assembled condition through suitable mechanical locking features. For example, the shuttle 16 may be provided with a detent 16n and the distal section provided with a slot 14n which engage upon full advancement of the shuttle within the distal section. It will thus be realized that the shuttle 16 is fixed to the distal section 12.

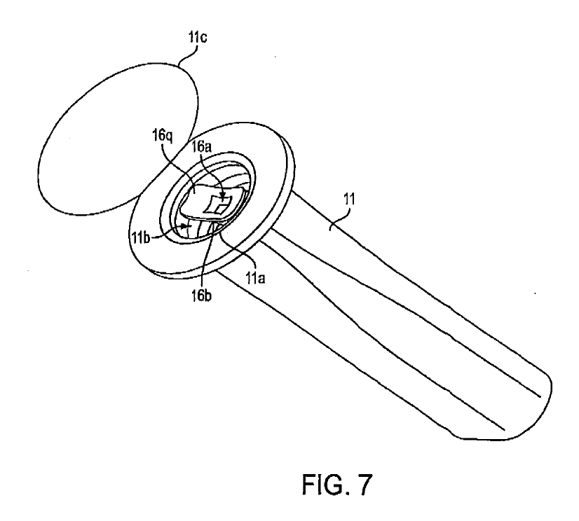

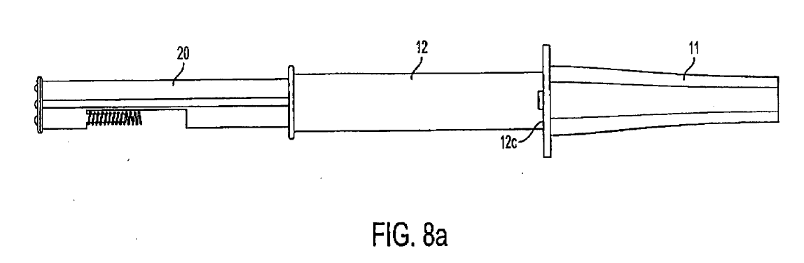

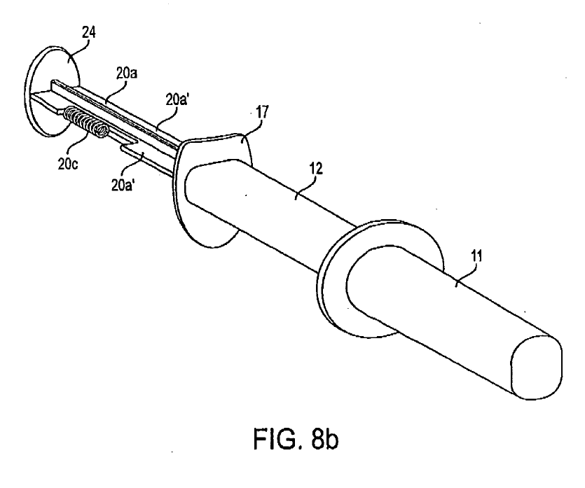

As stated above, the shuttle 16 and IOL 30 are positioned in the distal section 14 at manufacturing and then placed in a dry package or a vial of storage solution for storage and delivery to the surgeon. For wet packaging, to ensure storage solution reaches the IOL 30, the shuttle and distal section may each include one or more through-holes 14p, 16p which are in open communication with the IOL 30. One of many possible embodiments of a vial is seen in FIGS. 7 and 8a,b, wherein a vial 11 having an open end 11a and an internal cavity 11b is provided to accept the distal section 14 and shuttle 16 with the shuttle proximal end 16c thereof lying adjacent the open end 11a of the vial. One or more longitudinally extending fins or other similar features (not shown) may is be formed on the inside surface of vial 11 to align and maintain the distal section 12 at the desired orientation within vial 11. A rigid cover or a flexible cover sheet such as a foil seal 11c is attached to open end 11a to seal the vial. Seal 11c may be tethered to vial 11 by a flexible hinge (not shown) if desired. This feature keeps the seal with the vial after vial opening and thereby prevents having a ‘loose’ part in the operating suite. At the time of surgery, the package or vial 11 and distal section 12 are removed from any outer packaging in a sterile field and the vial cover seal 11c is removed to open vial 11 and access distal section 14 and shuttle 16. The proximal section 12 is likewise removed from its packaging in a sterile field. The nurse or surgeon proceeds to assemble the proximal and distal sections together by inserting the distal end 12c of the proximal section 12 into the open end 11a of the vial 11 (see FIGS. 5a,b). With the distal section 14 and shuttle 16 still in the vial, the proximal section open end 12c telescopes first over the shuttle proximal end 16b and then also over the distal section proximal end 14b. It is noted that the shuttle 16 may be provided with a proximal flange 16q at proximal end 16b to assist in maintaining proper alignment between the proximal section passageway 12a and the shuttle 16. Flange 16q may or may not touch the inner wall surface defining proximal section passageway 12a.

Upon further pressing of proximal section 12 against distal section end 14b results in the two sections attaching together. Various mechanical connection features may be employed to permit the quick and easy attachment of the proximal section 12 to the distal section 14 by simply pressing the two sections together as described above. Such features may include cooperating detents and recesses or a friction fit between the two sections, for example. In the embodiment shown in the Figures, a pair of detents 14d,e (FIGS. 6a-d) are provided on the outer wall surface of distal section 14 which align with and engage a pair of through-holes 12d,e formed on proximal section 12 adjacent open distal end 12c thereof (FIGS. 3a,b). When the proximal section 12 is pressed against the distal section 14, the detents 14d,e engage the through-holes 12d,e, respectively, and the sections become attached together. A radial flange 14f may be provided on distal section 14 to act as a stop against further advancement of the proximal section 12 on the distal section 14, i.e., to prevent advancement beyond the point of detent engagement. Once the proximal and distal sections have attached together, the proximal section 12 is lifted away from the vial 11 to remove the distal section (together with shuttle 16 and IOL 30 still coupled thereto) from the vial 11. The vial 11 and storage solution (or dry package) may then be discarded or recycled. The assembly of the injector device is now complete and the surgeon may proceed to inject the IOL 30 into a patient's eye by inserting rip 14c into an incision formed in the eye and pressing plunger 20 to advance the IOL 30 through and out the nozzle tip 14c (see FIG. 2; the eye not shown for sake of clarity).

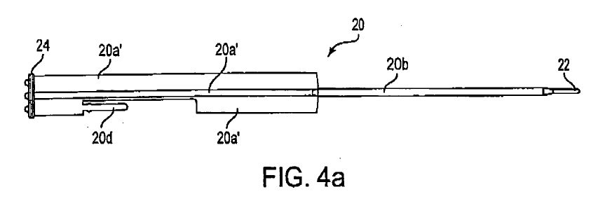

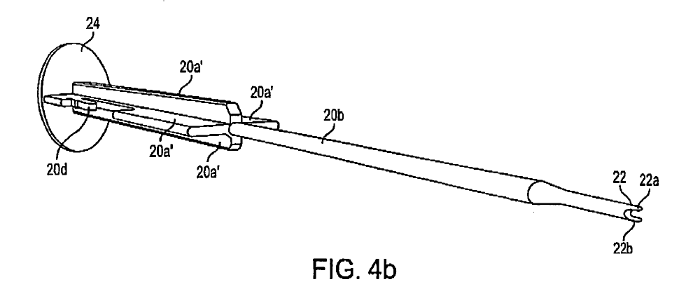

Referring to FIGS. 4a,b, it is seen that the plunger 20 includes distal and proximal plunger shaft lengths 20a, 20b, respectively, having a plunger tip 22 at the distal end thereof and a thumb press 24 at the proximal end thereof for manually operating the injector device. The plunger tip 22 is configured for engaging the IOL optic 31 at the periphery 31a thereof as the plunger 20 is advanced toward the distal tip 14c of distal section 14. It is very important that the plunger tip 22 not damage the IOL optic 31. The plunger tip 22 is thus designed to prevent damage to the IOL optic 31. In the preferred embodiment, the tip is bifurcated into first and second tip portions 22a and 22b, whereby the IOL optic periphery 31a becomes engaged between tip portions 22a, 22b as seen in FIG. 2B. It is understood that other plunger tip designs may be used with the present invention as desired. It is furthermore preferred that the plunger shaft is rotationally fixed within passageway 12a to prevent unexpected rotation of the shaft (and thus the tip 22) therein. For example, the plunger shaft may be rotationally fixed by forming the proximal shaft length 20b and passageway 12a non-circular in cross-section as shown.

In a particularly advantageous embodiment, the proximal length 20b of the plunger shaft is provided with one or more elongated flanges 20a′ which align with a like number of slots 12a′ formed between radially extending fins 21a-d formed on the inner wall surfaces of proximal section 12 adjacent proximal end 12b thereof (FIG. 5c). The purpose of flanges 20a′ and slots 12a′ is to provide tactile resistance therebetween and thereby allowing the surgeon more precise control and feel when advancing the plunger. The fins 21a-d may be made flexible yet resilient to provide the amount of tactile resistance desired. It is understood that other ways of providing tactile resistance between the plunger and injector body are within the scope of this invention. This provides the surgeon with continuous tactile feedback allowing the surgeon to advance the plunger (and thus the IOL) through the injector device in a very concise and controlled manner. Additionally, the flanges 20a′ and slots 12a′ help provide proper centering of the plunger shaft 20 and tip 22 relative to axis X_X along which the passageways of the components lie as explained above. Upon full advancement of the plunger, it is desirable to have the plunger automatically retract to some degree upon release of finger pressure against plunger finger press 24. In this regard, a spring 20c may be provided on a finger 20d on shaft length 20a. As the plunger is advanced, the spring 20c will interact with the one or more of the fins 21a-d as the plunger 20 is advanced therethrough.

When it is time to use the injector device, the surgeon selects a package or vial 11 having the appropriate IOL style and power preloaded in the shuttle and distal section stored in the vial as described above. The outer packaging is removed in a sterile field of the surgical suite. The proximal section having the plunger coupled thereto is also removed from its associated packaging in the sterile filed. The nurse or surgeon then attaches the proximal section 12 to the distal section 14 located in the vial in the manner described above. Once the proximal and distal sections 12,14 are attached together as shown in FIG. 2, the surgeon inserts the distal tip 14c into an incision cut into the eye and begins advancing the plunger 20. As the plunger 20 is advanced, the plunger tip 22 engages the optic periphery 31a and pushes IOL 30 forwardly. Upon continued advancement of the plunger 20, the IOL 30 is pushed through the shuttle passageway 16a and is expressed from distal tip 14c and into the eye (FIG. 2). As stated above, the spring 20c provides increasing bias in the reverse direction as the plunger reaches the fully advanced position. This occurs as spring 20c is compressed against one or more of the fins 21a-d. This assists the surgeon in maintaining precise control over plunger (and hence IOL) advancement and allows automatic retraction of the plunger upon relieving the pushing pressure being exerted against the plunger thumb press 24. This is useful for easily executing a second stroke of the plunger in order to engage and manipulate the trailing haptic into place in the eye. This feature, together with the bifurcated plunger tip 22, allows a more precise control and manipulation of the IOL with the plunger tip in-situ than would be possible with an injector device not having these features.

As discussed above, the device may be used for IOLs of any type and style. The configuration of the various component parts may likewise vary to accommodate the particular IOL style being employed with the device. Another embodiment of distal section 14′ and shuttle 16′ is seen in FIGS. 9a-c for holding an IOL 30′ having open loop haptics 30a′ and 30b′ extending from optic 31′. This configuration of shuttle 16′ includes a longitudinal passageway 16a′ extending between proximal end 16b′ and 16c′, respectively. The shuttle is divided into two longitudinal sections 16d′,e′ which are hinged together about living hinge 16k. In the open condition of shuttle 16′ seen in FIG. 9a, IOL loading area 16g′ is accessible to position IOL 30′ thereon, on section 16d′, for example. IOL loading area 16g′ opens into and communicates with longitudinal passageway 16a′ which is formed when shuttle 16′ is in the closed condition seen in FIGS. 9b,c. In this regard, registration pins 16h′ may be provided to engage holes 16i′ on sections 16d′, 16e′ to assist in aligning and correctly closing shuttle 16′. Various IOL placement features such as curved radius 16j′ and alignment pin 16k′, for example, may be formed on one or both sections 16d′, 16e′ to assist in proper placement of IOL 30′ in IOL loading 16g′.

Referring to FIG. 9c, the distal section 14′ may likewise vary in configuration to accommodate the configuration of shuttle being used. In this embodiment, distal section 14′ includes a longitudinal passageway 14a′ extending between proximal and distal ends 14b′, 14c′, respectively. Furthermore, one or more fingers 14d′; 14e′ extend from proximal end 14b′ and include a catch or other feature 14e″, 14f″ at the terminal end thereof to engage with the proximal end 16b′ of shuttle 16′.

Mechanical locking features such as one or more detents 14f′, 14g′ may be formed on the outer surface of fingers 14d′, 14e′ to engage an associated recess or slot 12d, 12e formed on the proximal section 12.

It may thus be realized that the present invention provides an injector device method and apparatus that may be provided in a variety of embodiments. The present invention is therefore not to be limited by the embodiments shown and described herein.

What is claimed is:

1. A method of packaging an intraocular lens in a portion of an intraocular lens injection device comprising the steps of:
a) providing an injector device having a distal section;
b) providing a shuttle and positioning the intraocular lens therein;
c) attaching said shuttle to said distal section;
d) depositing said shuttle, intraocular lens and distal section in a vial containing storage solution and sealing the vial.

2. The method of claim 1, and further comprising the step of ejecting the intraocular lens from the injection device by providing a proximal section and a plunger slidably received in said proximal section, opening the vial, attaching the proximal section to the distal section and removing the distal section, shuttle and intraocular lens together from the vial, and advancing the plunger which extends through said distal section and said shuttle and thereby ejects the intraocular lens from the injector device.

3. A method of packaging and subsequently preparing an injector device for use, said injector device adapted to receive, store and ship an intraocular lens in a portion thereof, said injector device reconfigurable from a storage condition to an injection condition, said method comprising the steps of:
a) providing a proximal section having a longitudinal passageway;
b) providing a distal section having a longitudinal passageway;
c) providing a shuttle having a longitudinal passageway, positioning an intraocular in said shuttle, and positioning said shuttle and intraocular lens together inside said distal section;
d) providing a vial having an open end leading into an internal cavity and dispensing a quantity of aqueous solution in said internal cavity;
wherein when said injector device is in the storage condition, said shuttle, said intraocular lens and said distal section are positioned and sealed in said vial of aqueous solution, and
wherein reconfiguring said injector device from said storage condition to said injection condition comprises the steps of:
opening said vial;
attaching said proximal section to said distal section; and
lifting said proximal section away from said vial and thereby removing said distal section, said shuttle and said intraocular lens from said vial.

4. An injector device adapted to receive, store and ship an intraocular lens in a portion thereof, and reconfigurable from a storage condition to an injection condition, said injector device comprising:
a) a proximal section having a longitudinal passageway;
b) a distal section having a longitudinal passageway;
c) a shuttle having a longitudinal passageway, said shuttle being adapted to hold an intraocular lens therein, said shuttle and intraocular lens being positioned inside said distal section; and
d) a vial having an open end leading into an internal cavity adapted to hold a quantity of aqueous solution and a closure for removably sealing said open end,
wherein when said injector device is in the storage condition, said shuttle, said intraocular lens and said distal section are positioned and sealed in said vial of aqueous solution and when said injector device is in said injection condition, said proximal section is attached to said distal section.

5. The device of claim 4 wherein said proximal and distal sections are colinearly attached and said distal section includes a distal tip at the end thereof opposite said proximal section and wherethrough said intraocular lens is expressed, and further comprising the step of using the injector device comprising the steps of:
providing a plunger in telescoping relation within said proximal and distal sections; and
advancing said plunger entirely through said distal section and thereby expressing said intraocular lens from said distal tip.

6. The device of claim 4, wherein said proximal section, said distal section and said shuttle each include a longitudinal passageway which all lie along a common axis in said injection condition of said injector device.

7. The device of claim 4 and further comprising the step of packaging said proximal section separately from said vial.

8. The device of claim 4 wherein said shuttle and said distal section are coaxially arranged.

9. The device of claim 4 wherein said shuttle is attached to said distal section in coaxial relationship.

10. The device of claim 4 and further comprising the step of sterilizing said shuttle, said intraocular lens, and said distal section when sealed in said vial.

11. The device of claim 10 wherein said sterilization step is steam sterilization.

12. The device of claim 10 wherein said shuttle is snap fit to said distal section.

13. The device of claim 10 wherein said proximal section and said distal section are snap fit together in collinear relationship when in said injection condition.

14. An injector device component preloaded with an intraocular lens comprising:
a) a distal section;
b) a shuttle adapted to receive an intraocular lens therein, said shuttle being attached to said distal section; and
c) a vial containing a storage solution wherein said shuttle, intraocular lens and distal section are positioned and sealed in the vial.

15. The injector device component of claim 15 and further comprising a proximal section component adapted to be attached to said distal section in colinear relationship.

16. The injector device component of claim 15 wherein said shuttle and said distal section each include one or more through-holes allowing fluid communication of said storage solution with said intraocular lens in said shuttle.

17. The injector device of claim 15 wherein said intraocular lens is positioned in said shuttle in an unstressed condition.
